(19) United States
(12) Patent Application Publication
(10) Pub. No.: US 2011/0054428 A1
(43) Pub. Date: Mar. 3, 2011

(54) Male Urethral Retention Device for Absorbing Liquid Discharge

(76) Inventor: Alexander Hill, Bradley, IL (US)

(21) Appl. No.: 12/871,235

(22) Filed: Aug. 30, 2010

Related U.S. Application Data
(60) Provisional application No. 61/275,467, filed on Aug. 31, 2009.

(51) Int. Cl. A61F 5/44

(52) U.S. Cl. 604/349

(57) ABSTRACT

A flexible sheath used for absorbing and retaining liquid discharge from a male penis, and in particular, for male urinary incontinence and for use after intercourse. The device comprises an opening, a single interior cavity, an upper elastic band, and an inner fluid absorbent material. The opening allows for insertion of the penis or shaft, and the single interior cavity houses the penis and shaft. The elastic band is positioned to keep any excess liquid discharge from escaping the inner fluid absorbent material. A water resistant coating on the outside of the flexible elastic woven sheath also keeps unabsorbed liquid discharge from escaping. This invention may also be useful to catch and absorb condensate or liquid squirts on water pressure testing locations on the inlets and outlets of HVAC water coils.

This application claims priority to provisional patent application Ser. No. 61/275,467 entitled “Uricatch 2Go,” filed Aug. 31, 2009, to the extent allowed by law.

### BACKGROUND OF THE INVENTION

1. Field of the Invention

The present invention relates to a liquid retaining device for containing liquid discharge from the penis, and in particular, for male urinary incontinence and for use after intercourse.

2. Description of the State of the Art

Urethral discharge retention devices for absorbing liquid discharge are used to eliminate the discomfort and embarrassment caused by wet spots on pants after urination or intercourse. Knowing that spots won't show on a man's pants after urination gives men the peace of mind needed to walk back into that business meeting, walk confidently toward his partner on a date, or stand comfortably across from his friends during a conversation. Fluid retention devices are particularly beneficial to older males, as well as men with prostate problems. This device could also be worn after sexual intercourse to absorb leaking fluids. Finally, in a completely different field, this device maybe useful to catch and absorb condensate or water squirts on water pressure testing locations on the inlets and outlets of HVAC water coils.

Currently, certain urethral discharge retention devices that are presently available are formed as undergarments, such as diapers. This type of urethral discharge retention device can be uncomfortable and may not fit well underneath pants or jeans. Also rashes, and in particular, diaper rashes are a potential unintended by-product of the diaper-like device. Furthermore, while a diaper may prevent the embarrassment of liquid spots from forming on the front of pants, the bulging nature of the diaper's absorbing material could create embarrassment due to the unusual fit underneath the pants or jeans.

Another type of urethral discharge retention device fits around the tip of the penis to catch the discharge. These devices are generally in a bag shape or contain an outer shell to prevent liquid from seeping through. The bag shape, however, is sometimes difficult to keep attached easily to the penis, having a tendency to slide off. Also, an outer shell might be uncomfortable and may cause other variations of penile embarrassment different from wet spots on pants or jeans.

There are several presently available urethral discharge retention devices that provide a means to absorb liquid discharge from the penis. See for example, U.S. Pat. No. 4,601,716 (the '716 patent); U.S. Pat. No. 2,873,740 (the '740 patent); and U.S. Pat. No. 4,668,229 (the '229 patent).

The 716' patent discloses a sheath with a moisture absorbent pad to absorb excretions and urine. Specifically, the '716 patent discloses a sheath formed as a laminate structure having an outer moisture resistant sheath and an inner, coextensive, moisture permeable liner, with an intermediate moisture absorbent pad. Thus, the inner liner with the moisture absorbent pad lines the entire inner cavity of the sheath invention. The sheath seals the head of the penis to prevent fluid from escaping, and secures to the penis by either an elastic band closure around the neck or a Velcro type fabric attachment band. The '716 patent fails to disclose a flexible sheath for collecting and retaining fluid discharge from an opening in a liquid-conveying shaft, where the outer sheath comprises a single flexible piece, and a cavity is formed with a first section of the cavity extending a pre-determined distance from a closed end. This configuration allows for absorption of fluid while reducing the cost incurred in lining the entire cavity with fluid absorbing material. Furthermore, the '716 patent fails to disclose an elastic band located at the termination location of the inner sheath of fluid absorbing material. The '716 patent limits the location of a band to the opening of the device. Thus, the '716 patent's band fails to provide a means for securing an inner sheath of absorbing material and a means of keeping fluid from escaping the sheath.

The '740 patent discloses a hard sheath to prevent liquid discharge from escaping the device. Specifically, the sheath, fits over the head and shaft of the penis and is lined inside with an absorbent liner to absorb the fluid. Furthermore, the '740 patent discloses a belt which wraps around the user's leg or waist, for example, so that the device maybe utilized properly. The '740 patent fails to disclose a flexible sheath for collecting and retaining fluid discharged from an opening in a liquid-conveying shaft for absorbing fluid discharge and fluid accumulation, preventing contact between the fluid and undergarments or pants. The flexible elastic woven construction of an embodiment of the presently claimed invention prevents the device from falling off or coming loose. The '740 patent teaches securing the sheath using a belt instead of the sheath material itself. Furthermore, the '740 patent fails to disclose an elastic band attached to an outer circumference portion of the flexible fluid-resistant sheath, preventing the escape of discharged fluid.

The '229 patent discloses a sheath and loop made of a non-resilient and non-elastomeric material. Specifically, the sheath provides a means to keep liquid discharge from making contact with undergarments or pants. Additionally, a loop, which attaches to the testicles or penis shaft, secures the sheath to the user. Furthermore, the '229 patent discloses a slot on the head side of the penis so that a user can urinate without removing the sheath. The '229 patent fails to disclose a flexible sheath for collecting and retaining fluid discharged from an opening in a liquid-conveying shaft for absorbing fluid discharge and fluid accumulation. The flexible elastic woven construction of an embodiment of the presently claimed invention prevents the device from falling off or coming loose. The '229 patent teaches securing the sheath using a loop instead of the sheath material itself. Additionally, the '229 patent teaches the implementation of a slot so that a user may urinate without removing the device. This feature creates the potential for fluid to seep through the slot of the device and onto the pants or undergarments of the user. This is a primary problem that the presently claimed invention prevents.

### SUMMARY OF THE INVENTION

The present invention provides, in one embodiment, a flexible sheath that may be disposable. The flexible sheath may be made of an elastic woven material, and has an opening, a single interior cavity, an inner sheath, and an outer elastic band. A liquid resistant component covers the exterior of the sheath to prevent liquid from escaping out of or entering into the sheath. The opening of the sheath is designed so that a shaft, and a head at one end of the shaft, can be inserted through the opening and into the single interior cavity. The single interior cavity has two sections, the head section and the shaft section. The head section is adapted to house at least a portion of the head located at one end of the shaft. The shaft section houses at least a portion of the shaft. The inner fluid absorbing sheath is located inside the single interior cavity coextensively with the head section. The inner sheath absorbs the fluid discharge or fluid accumulation. The upper elastic band snuggly secures the soft elastic woven sheath to the shaft and the head at one end of the shaft. The present invention provides a means for men with urinary incontinence to absorb urinary discharge preventing discomfort and the embarrassment of wet marks on pants or undergarments. Furthermore, the present invention may be used after intercourse to avoid the discomfort or embarrassment of wet marks on pants or undergarments from residual penile discharge.

### BRIEF DESCRIPTION OF THE DRAWINGS

The following figures are included to illustrate the embodiments of the devices and techniques of the present invention. In these figures, like numerals represent like features in the several views. It is to be noted that these figures, illustrating the several views of the present invention, unless stated to be otherwise, are not necessarily drawn to scale. Nor are the contents of the drawings to be considered as limiting the scope of the present invention as defined in the claims.

FIG. 1 is a perspective view of an embodiment of the retention device, which depicts the outer sheath, elastic band, the opening, and a shaft inserted into the retention device;

FIG. 2 is a cross-sectional view of an embodiment of the retention device with the shaft removed, which depicts the outer sheath, inner sheath, and single interior cavity; and

FIG. 3 is a cross-sectional view of an embodiment of the retention device in use.

### DETAILED DESCRIPTION OF THE ILLUSTRATED EMBODIMENTS

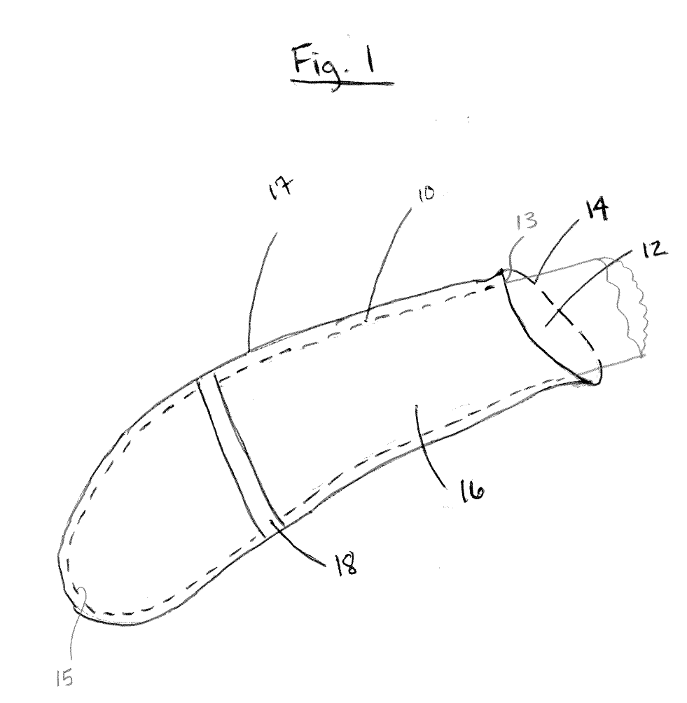

Referring to the embodiment in FIG. 1, a flexible sheath 10 has a single interior cavity 12, and an opening 14 communicating with cavity 12. The opening 14 is at one end of the flexible sheath 10 allowing for entrance of a shaft 16 having a head 15 at one end of the shaft 16. The single interior cavity 12 houses the shaft 16 and the head 15 disposed at one end of the shaft 16 when in use. The flexible material of the sheath 10 allows the device to fit securely and comfortably around the penis of the user so that the user doesn't have to worry about the device falling off or irritating him. In the illustrated embodiment, the sheath 10 is made of a flexible elastic woven material, but other flexible materials as are known in the art can be used. The flexible elastic woven material also provides a seal 13 with the shaft 16 at opening 14 to prevent fluid from exiting the opening 14. The flexible elastic woven sheath 10 includes a liquid resistant component, shown as a cover 17, covering the exterior of the sheath 10 to prevent liquid from escaping out of or entering into the sheath 10. The flexible elastic woven sheath 10 may be disposable.

An elastic band 18 is fixedly attached circumferentially to the flexible sheath 10 so that the single interior cavity 12 snuggly engages at least a portion of the head at one end of the shaft 16. The elastic band 18 provides an extra precaution to ensure that the flexible sheath 10 remains on the user when used, as well as providing an additional seal to keep fluid from exiting the opening 14 of the sheath 10.

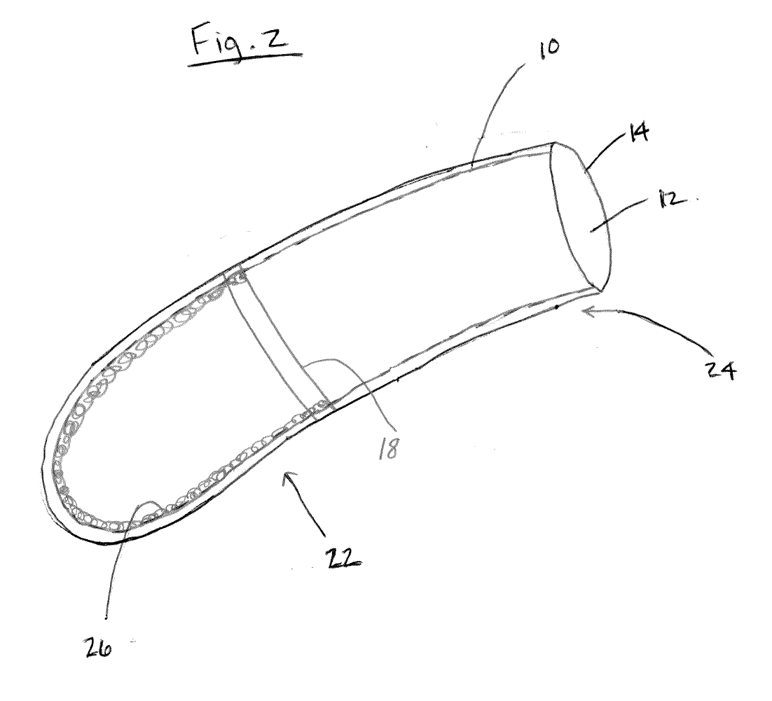

Referring to FIG. 2, this cross-section view depicts the flexible single interior cavity 12 of the flexible elastic woven sheath 10. The single interior cavity 12 has two sections, the head section 22 and the shaft section 24. The head section 22 houses at least a portion of the head 15 at one end of the shaft 16 when the shaft 16 is inserted into cavity 12. The shaft section 24 houses at least a portion of the shaft 16.

An inner sheath 26 made of a fluid absorbent material extends coextensively with the head section 22 of the single interior cavity 12, and does not extend the full length of flexible sheath 10. The inner sheath 26 absorbent material is similar to the material used for absorbing fluid in diapers. Typically, this absorbent material is of a cellulose material such as air laid wood pulp fluff. The absorbent material may also comprise a coform material composed of an air laid mixture of cellulosic fibers and synthetic polymer fibers. In addition, the absorbent material may optionally include natural or synthetic superabsorbent materials, such as pectin, carboxymethyl cellulose, guargum, polysaccharides, cross-linked synthetic polymers and the like. For example, polymers composed of alkali metal salts of lightly cross-linked plyacrylic acid have been found to be suitable superabsorbent materials. The superabsorbent materials are available from suppliers such as Stockhausen, Hoechst Celanese, and Dow Chemical Co. The absorbent material forming inner sheath 26 can include a tissue wrap to increase the material's structural integrity. Furthermore, the absorbent material of inner sheath 26 can be made of a wood pulp fluff integrated with a thermo-plastic absorbent material treated with a surfactant. Thermo binders, such as Pulpex® can be used in blends or layering with fluff and superabsorbent material.

The head section 22 of woven sheath 10 is positioned so that it begins at the end opposite the opening 14. The head section 22 ends where the shaft section 24 begins. The shaft section 24 of the sheath extends to the opening 14. The position where the head section 22 ends and the shaft portion 24 begins may vary depending on the intended use for the flexible elastic woven sheath 10. This allows for the effective and efficient use of the inner sheath 26 that is coextensive with the head section 22 and composed of absorbent material. Typically, the head section 22 extends approximately 1 inch from the end opposite the opening 14 along the flexible elastic woven sheath 10 when in use.
i. The elastic band 18 extends circumferentially around sheath 10 at a location adjacent the extent of absorbent material 26, as shown in FIG. 2, which location also corresponds to where the head section 22 and the shaft portion 24 of sheath 10 meet. The elastic band 18 thus provides a seal between absorbent material 26 and shaft 16 when shaft 16 is inserted into sheath 10.

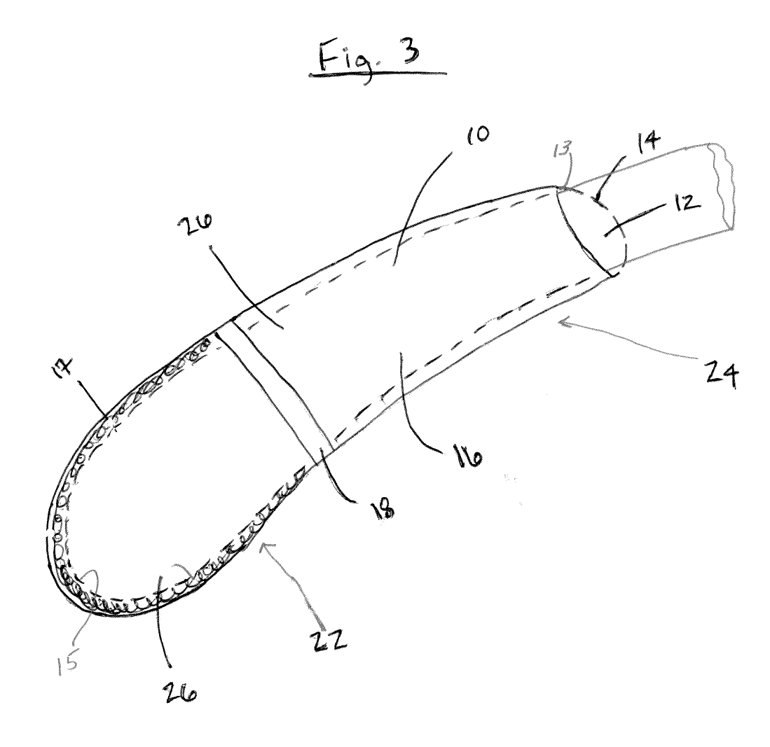

Referring to the embodiment of FIG. 3, a cross-sectional view shows the flexible elastic woven sheath 10 in use with shaft 16 inserted into cavity 12. The shaft 16 with the head 15 at one end of the shaft 16 is inserted through the opening 14 into the single interior cavity 12. When liquid is discharged from the head portion 15 of the shaft 16, the absorbent material that comprises the inner sheath 26, extending the length of head section 22 absorbs the liquid discharge. Additionally, any fluid not completely absorbed by the inner sheath 26, is absorbed by the flexible elastic woven sheath 10. The fluid is ultimately contained by the absorbent material 26, and possibly by the flexible elastic woven sheath 10 by the liquid resistant component 17 covering the exterior of the sheath 10 and the elastic band 18. The elastic band 18 is located along the length of the flexible elastic woven sheath 10 at a location most effective to carry out its functions described above. For example, the upper elastic band 18 can be positioned where inner sheath 26 ends at the beginning of the shaft section 24. This forms a seal between the inner sheath 26 and the shaft 16. Once the flexible elastic woven sheath has completed its function, the shaft 16 and the head 15 at one end of the shaft 16, are removed from the single interior cavity 12 through the opening 14, and the woven sheath 10 is properly discarded.

The foregoing description of the illustrated embodiment of the invention has been presented for purposes of illustration and description, and is not intended to be exhaustive or to limit the invention to the precise form disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims set forth below.

I claim:

1. A flexible sheath for collecting and retaining fluid discharge from an opening in a liquid-conveying shaft, the flexible sheath adapted to be fitted over and cover the opening of the shaft and to receive and retain fluid discharged from the opening in the shaft, the flexible sheath comprising:
a. an outer sheath comprising a flexible, fluid resistant material, the fluid resistant material defining a cavity and an opening communicating with the cavity at one end of the fluid resistant material and a closed end of the cavity formed at an opposite end from said opening;
b. the cavity having a first section extending a pre-determined distance from the closed end in a direction towards said opening, said first section having a termination location prior to reaching said opening, said first section of said cavity adapted to receive a first head portion of said shaft;
c. the cavity having a second section extending from said termination location of said first section and extending toward said opening; said second section adapted to receive a second portion of said shaft;
d. an inner sheath of fluid absorbing material disposed in said cavity, said inner sheath extending coextensively from said termination location of said first section to a closed end of said inner sheath, said closed end of said inner sheath located adjacent the closed end of the flexible sheath;
e. said inner sheath forming an additional cavity, said additional cavity adapted to receive the first head portion of the shaft when the first head portion of the shaft is inserted into the opening;
f. an elastic band attached to an outer circumference portion of said flexible fluid-resistant material, said outer circumference portion located adjacent said termination location of said inner sheath of fluid absorbing material; said elastic band adapted to snuggly engage a portion of the fluid absorbing material against the head portion of the shaft and prevent the escape of discharged fluid from the fluid absorbing material.

2. The flexible sheath of claim 1, wherein:
the outer sheath comprises a single piece of elastic woven flexible fluid resistant material.

3. The flexible sheath of claim 2, wherein the outer sheath further comprises a liquid resistant component surrounding the outer sheath.

4. The flexible sheath of claim 1, wherein:
the liquid-conveying shaft is a male mammalian penis.

5. The flexible sheath of claim 1, wherein:
said flexible outer sheath and said inner sheath are disposable.
